(19) United States
(12) Patent Application Publication
(10) Pub. No.: US 2020/0126678 A1
(43) Pub. Date: Apr. 23, 2020

(54) METHOD OF CREATING AN ARTIFICIAL INTELLIGENCE GENERATED DIFFERENTIAL DIAGNOSIS AND MANAGEMENT RECOMMENDATION TOOL BOXES DURING MEDICAL PERSONNEL ANALYSIS AND REPORTING

(71) Applicants: David Douglas, Winter Park, FL (US); Kathleen Douglas, Winter Park, FL (US); Robert Douglas, Winter Park, FL (US)

(72) Inventors: David Douglas, Winter Park, FL (US); Kathleen Douglas, Winter Park, FL (US); Robert Douglas, Winter Park, FL (US)

(21) Appl. No.: 16/597,910

(22) Filed: Oct. 10, 2019

Related U.S. Application Data
(60) Provisional application No. 62/850,002, filed on May 20, 2019.
(60) Provisional application No. 62/748,555, filed on Oct. 22, 2018.

(51) Int. Cl. G16H 80/00; G06F 17/24; G06N 3/08; G06N 20/00; G16H 30/40; G16H 20/10

(52) U.S. Cl. CPC G16H 80/00; G06F 17/248; G06N 3/08; G16H 50/20; G16H 30/40; G16H 20/10; G06N 20/00

(57) ABSTRACT

Techniques for precision reporting in medicine are disclosed. These techniques include assistance with generating a differential diagnosis and management plan, assured communication strategies and error prevention in reporting.

### TECHNICAL FIELD

Aspects of this disclosure are generally related to the development of a differential diagnosis and management plan in medicine.

### BACKGROUND

To generate a differential diagnosis, physicians must synthesize information about the patient such as age, past medical history and current clinical presentation. Sometimes the pieces of information are gathered accurately, but a synthesis errors occurs. Another type of error includes the failure to specifically look for additional secondary findings (e.g., pertinent positives and pertinent negatives). Another type of error is failure to communicate a critical finding to another physician. Another type of error is wherein the findings are incongruent with the conclusion.

### SUMMARY

All examples, aspects and features mentioned in this document can be combined in any technically conceivable way.

In accordance with an aspect a method comprises: a method utilizing artificial intelligence during medical personnel analysis and reporting to generate a differential diagnosis and, if applicable, the associated management recommendations to improve patient care comprising: generating information fields; inputting patient specific information (e.g., data elements) within the information fields; performing an artificial intelligence (AI) program; outputting a differential diagnosis and, if applicable, management recommendation(s) by the AI; reviewing of the AI generated differential diagnosis and management recommendation(s) by the human; if applicable, modifying the patient specific information fields, the differential diagnosis and management recommendation(s); and, updating the training dataset. The preferred embodiment of this is for the radiologist. During the radiologist's reporting program, several data elements are inputted into the report. These include the type of scan, field of view, organs included in the imaged volume, patient history, imaging findings within the field of view, etc. The pathology can be extremely complex with a very large range of tumors, infections, vascular abnormalities, etc. The neuroradiologist deals with over 1000 different pathologies on a brain MRI scan. A source of error may include forgetting to include a particular diagnosis in the differential diagnosis that is reported. As an example, the radiologist might describe “leptomeningeal enhancement” and include bacterial meningitis, leptomeningeal carcinomatosis (spread of cancer along the surface of the brain), and sarcoidosis (an inflammatory program), but forget to include tuberculosis. This is a significant source of potential error in the radiology reporting system. If the radiologist misses the diagnosis, often the patient will go untreated and may suffer terrible health consequences. Therefore, implementing a real-time, artificial intelligence (AI) driven differential diagnosis tool box which reads the radiologist's report in real time and produces a working differential diagnosis for the radiologist to reference may prove extremely helpful. The radiologist only thought of 3 diagnoses. The AI would have identified 4 diagnoses. Therefore, the differential diagnosis tool box can avert the error of omitting a key differential diagnosis or the error of incorrect ordering of the likelihood of the diagnoses. For example, the AI system can include hyperlinks to key references to help teach the radiologist. Furthermore, the radiologist may think of the correct diagnosis list, but not provide the correct management recommendation. As an example, the radiologist may state “suspected scaphoid fracture”, but forget to state “recommend orthopedic consultation, immobilization and follow up imaging 10-14 days from the time of injury”. This can be averted through implementation of the management icon tool box. The process for a management icon would be similar to that of a differential diagnosis icon. For simplicity, this patent will primarily focus on the differential diagnosis icon, but the intent is for the system to have both icons.

In some embodiments, the method comprises artificial intelligence algorithms comprising deep artificial neural networks and other machine learning algorithms. The method comprises medical personnel reporting including diagnostic radiologists, primary care physician clinic notes or other medical personnel notes.

In some embodiments, the method comprises generating information fields comprising patient demographics, physical examination findings, laboratory findings, radiology scan type, radiology checklist item, radiology imaging findings, images or other patient specific features.

In some embodiments, the method comprises generating a list of terminology to be included in the information fields.

Some embodiments comprise creation of key words such that a communication note with referring clinician is required. A simple example of this would be to correctly describe acute epidural hematoma but forgetting to call the referring physician to notify of this critical information. This patent's system integrates artificial intelligence in the reporting so this error can be averted.

Some embodiments comprise creation of key words wherein a secondary terminology (e.g., pertinent positive, pertinent negative, measurement, etc.) is required. For example, if the word fracture is stated, the pertinent positive / pertinent negative list would include displacement, angulated, etc. Another simple example would be to state the word epidural hematoma but forgetting to measure the size of it. This patent's system integrates artificial intelligence in the reporting, so key words or numbers may trigger the mandatory inclusion of other key words or numbers. This can serve to further improve patient care.

In some embodiments, the method comprises inputting patient specific information comprises computer inputted information and human inputted information.

In some embodiments, the method comprises inputting patient specific information comprises utilizing single fields or combining multiple fields.

In some embodiments, the method comprises inputting patient specific information comprises selecting key items within a field such as numbers, single words or combinations of multiple words.

In some embodiments, the method comprises performing a machine learning program includes deep artificial neural networks and other machine learning algorithms.

In some embodiments, the method comprises performing a machine learning program includes utilization of materials including training datasets and medical references. In some embodiments, the method of machine learning includes generation and application of inclusion criteria resulting in a list of all possible differential diagnoses based one or more fields. In some embodiments, the method of machine learning includes generation and application of exclusion criteria where non-relevant differential diagnoses are eliminated from display. For example, if a chest imaging examination is performed and a key terminology in the lung field is mass and if the AI determines that glioblastoma multiforme is on the differential diagnosis, it would be excluded since glioblastomas only occur in the brain and therefore would be excluded. If child, exclude differential diagnoses that only pertain to the elderly. If order type is of a particular body part, then exclude body parts that would not be included in the field of view.

In some embodiments, the method of machine learning comprises generating the differential diagnoses based on information from two or more fields.

In some embodiments, the method of outputting a differential diagnosis by the machine learning algorithm comprises visual representation to the user via a pop-up box (e.g. tool box icon) on the computer or auditory representation such as an audible recording.

In some embodiments, the method comprises reviewing the differential diagnosis by the machine learning algorithm comprises human review of the pertinent item(s) considered by the algorithm and, if applicable, weighting factors, past medical reports and other medical references.

In some embodiments, the method comprises reviewing the differential diagnosis by the machine learning algorithm comprises selecting the diagnosis or differential diagnosis to be sent to the conclusion (aka, impression, assessment) section of the report. This could be accomplished by double clicking the diagnosis or differential diagnosis or drag-and-drop or similar type methods.

In some embodiments, the method comprises reviewing the management recommendations by the machine learning algorithm comprises selecting the management recommendation to be sent to the conclusion (aka, impression, assessment) section of the report.

In some embodiments, the method comprises utilization of the patient specific information fields, such that the differential diagnosis and/or the management recommendation changes in real time when at least one item the patient specific information changes in at least one information field.

In some embodiments, the method comprises providing a hyperlink to medical reference materials supporting the differential diagnosis and/or management recommendations provided.

In some embodiments, the method comprises updating the training dataset comprises adding new information including the patient specific information fields with an associated differential diagnosis and/or management recommendation.

In accordance with an aspect a method comprises: a method of assessing the congruency between data within a medical report and the conclusion of a medical report comprising: loading the medical report into a computer; performing an artificial intelligence algorithm based on the data within a medical report and, if applicable, other accessible medical data (e.g., via electronic medical record) to determine top differential diagnosis(es) and associated management recommendation(s); determining the similarities and/or differences in the differential diagnosis in the medical report and the differential diagnosis determined by the AI; if applicable, determining the similarities and/or differences in the management plan in the medical report and the management plan determined by the AI; presenting the results; if applicable, modifying either the medical data in the report, the differential diagnosis and/or the management plan. It is extremely important to have the findings be consistent with the differential diagnosis and the management plan. If the radiologist describes on a chest CT words like a phrase like “spiculated 3.5 cm mass in the right upper lobe of the lung” but then in the impression of the report states “pneumonia”, then the findings are incongruent with the impression. It would be discordant because the findings are describing lung cancer and the impression states pneumonia. The findings and impression are therefore discordant. The radiologist could be alerted to a discordant findings-impression prior to signing off the report via an alert, such as a pop-up box warning that the radiologist sees advising to resolve the discordance between the findings and impression prior to signing the report.

In accordance with an aspect a method of assessing the congruency between data within a medical report and the conclusion of a medical report comprises: loading the medical report into a computer; and performing an artificial intelligence program to determine the congruency between the patient specific information within the information field(s) and the impression section of the report. Some embodiments comprise alerting the medical professional of any discordance between the patient specific information within the information field(s) and the impression section of the report.

In accordance with an aspect, a method of precision radiology reporting comprises: generating a checklist for a radiology examination with multiple information fields; generating a list of terminology inappropriate for said information fields; entering text into information fields in the radiology report; and performing an automated review of said entered text wherein a notification is presented to the user if terminology inappropriate for said information field is identified.

A method of precision radiology reporting comprising: generating a checklist for a radiology examination with multiple information fields; generating a list of terminology for said information field(s) which require secondary descriptive terminology; generating a list of said secondary descriptive terminology; entering text into information fields in the radiology report; and performing an automated review of said entered text wherein a notification is presented to the user if a said terminology for said information field(s) which require secondary descriptive terminology is not accompanied by said secondary descriptive terminology.

In accordance with an aspect, a method of characterizing a radiology report comprises: generating a checklist for a radiology examination with multiple information fields; generating a list of preferred terminology preferred for said information fields; entering text into information fields in the radiology report; and performing an automated review of said entered text wherein a quantitative metric on the frequency of the preferred terminology entered in relation to the total text is presented to the user. This could be used as an efficiency number for reports. Some radiologists create reports that are so lengthy and so wordy that it is a hindrance to medical care. This type of metric would be useful to help improve efficiency in reporting.

In accordance with an aspect a method of assured communication of critical patient information during medical reporting comprises: generating a report template with information field(s); generating a comprehensive list of terminology indicating critical patient information and requiring communication for each information field in the report template; selecting user(s) to receive critical patient information; inputting patient specific information within the said information field(s); analyzing said patient specific information in said information field(s) for said comprehensive list of terminology indicating critical patient information and requiring communication; and implementing a digital alert notifying said critical patient information to said users. Some embodiments comprise electronic notification to a first user when a second user receives said digital alert.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 illustrates a method utilizing artificial intelligence during medical personnel analysis and reporting to generate a differential diagnosis and, if applicable, management recommendations to improve patient care.

FIG. 2 illustrates the method of utilizing artificial intelligence during medical personnel analysis and reporting to generate a differential diagnosis and to refine the differential diagnosis through the application of exclusion criteria.

FIG. 3 illustrates sample artificial intelligence algorithms including deep artificial neural networks and other machine learning algorithms.

FIG. 4 illustrates generating information fields comprised of patient demographics, physical examination findings, laboratory findings, radiology scan type, radiology checklist item, radiology imaging findings, images or other patient specific features.

FIG. 5 illustrates an example list of terminology to be included in the information fields.

FIG. 6 illustrates computer inputted information and human inputted information for the AI algorithm.

FIG. 7 illustrates utilization of materials for artificial intelligence algorithms.

FIG. 8 illustrates utilization of materials for artificial intelligence algorithms.

FIG. 9 illustrates visual representation to the user via visual representation or auditory representation.

FIG. 10 illustrates human review of the pertinent item(s) considered by the AI algorithm.

FIG. 11 illustrates human reviewing the differential diagnosis by the machine learning algorithm, selecting the diagnosis or differential diagnosis to be sent to the conclusion (aka, impression, assessment) section of the report.

FIG. 12 illustrates modifying the patient specific information field(s) and/or the differential diagnosis(es).

FIG. 13 illustrates updating the training dataset.

FIG. 14 illustrates a method of assessing the congruency between data within a medical report and the conclusion of a medical report.

FIG. 15 illustrates a method to detect inappropriate terminology in a report.

FIG. 16 illustrates a method to quantitatively assess report performance.

FIG. 17 illustrates a method of assured communication of critical patient information during medical reporting.

FIG. 18 illustrates a method to improve medical reporting by alerting a user when secondary descriptive terminology should be inputted.

### DETAILED DESCRIPTIONS

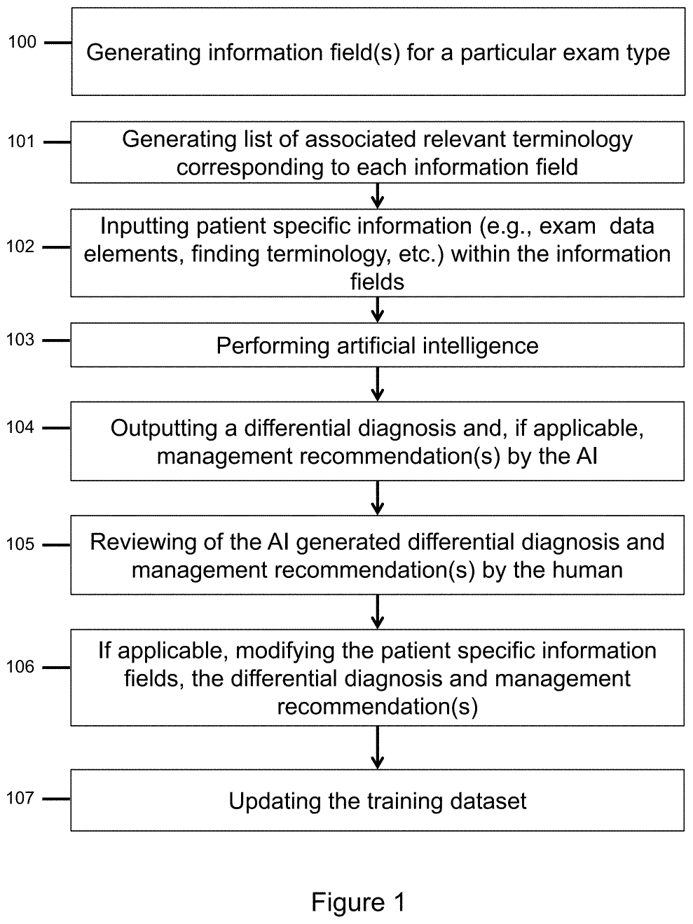

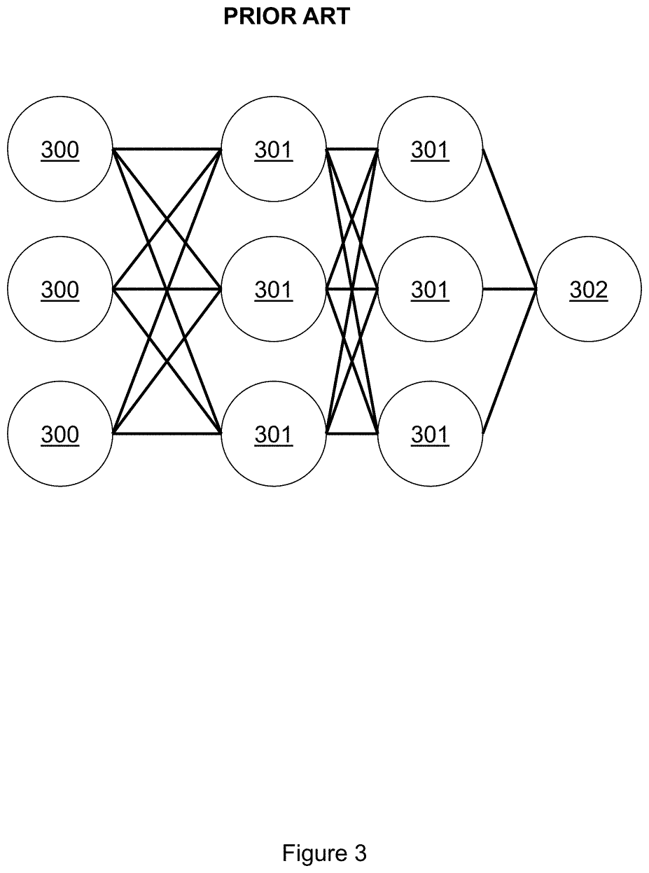

FIG. 1 illustrates a method utilizing artificial intelligence during medical personnel analysis and reporting to generate a differential diagnosis and, if applicable, management recommendations to improve patient care. The flow diagrams do not depict the syntax of any particular programming language. Rather, the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables, are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order. The first step 100 is to generate information field(s) for a particular exam type. For example, for a CT scan of the chest, an example information field would be “lungs”. The second step 101 is to generate a list of associated relevant terminology corresponding to each information field. For example, a list of relevant terminology for the information field “lungs” would include but is not limited to the following: “spiculated”; “nodule”; “mass”; “linear”; “solid”; “clear”; and, “sub-solid”. The third step 102 is to input patient specific information (e.g., data elements, finding terminology, etc.) within the information fields. At least some of the elements inputted should be on the list in step two 101 above. For example, within the information field “lung”, the patient specific information that could be inputted would be “1.5 cm round, pulmonary nodule with spiculated margins”. The terminology “nodule”, “solid” and “spiculated” are in the list of associated relevant terminology in step 2 above. This patient specific information could be inputted by the radiologist or alternatively through an artificial intelligence process. The fourth step 103 is to perform an artificial intelligence process. An example, artificial intelligence process is neural networks, such as is shown in FIG. 3. The fifth step 104 is to have the artificial intelligence process output a differential diagnosis and, if applicable, management recommendation(s). For example, the AI process would output “Most likely diagnosis is lung cancer. Recommend CT guided biopsy.” The sixth step 105 is for the radiologist to review the AI generated differential diagnosis and management recommendation(s) by the human. For example, the radiologist reviews the images, information field “lung”, patient specific information “1.5 cm round, pulmonary nodule with spiculated margins” and the AI generated differential diagnosis and management recommendations “most likely diagnosis is lung cancer. Recommend CT guided biopsy.” Then, in the seventh step 106, based on the radiologist's wisdom, if applicable, the radiologist modifies the patient specific information fields, the differential diagnosis and management recommendation(s). For example, the radiologist can agree with the AI generated differential diagnosis and management plan. Alternatively, the radiologist can disagree with the differential diagnosis and management plan and modify the recommendations. In the eighth step 107, the training dataset is updated accordingly.

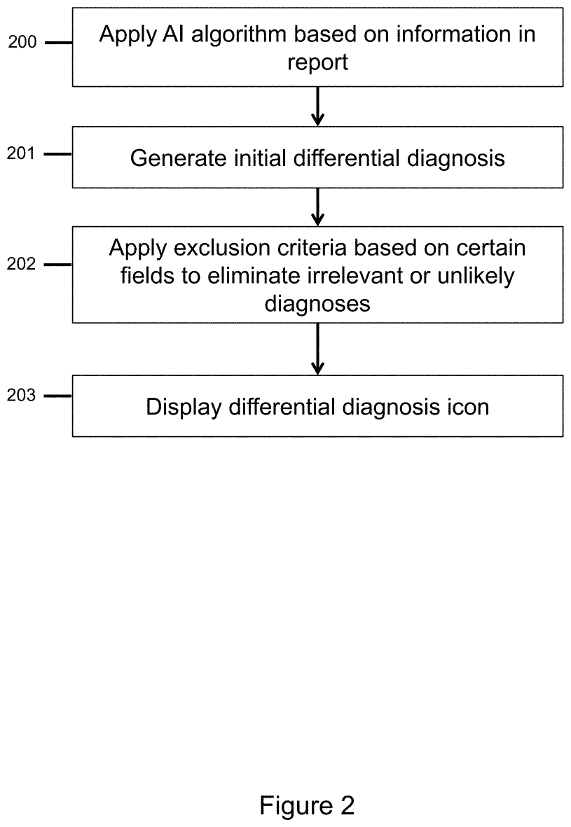

FIG. 2 illustrates the method of utilizing artificial intelligence during medical personnel analysis and reporting to generate a differential diagnosis and to refine the differential diagnosis through the application of exclusion criteria. The flow diagrams do not depict the syntax of any particular programming language. Rather, the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables, are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order. In the first step 200, the AI algorithm is applied based on the information in the report. There are two options at this juncture. First, the AI could be applied based on the entire report (i.e., every single word, number, etc.). Second, the AI could be applied based on only a subset of the elements in the report (e.g., only based on the information field “lung”, or combinations of fields, such as both the field “lung” and the field “heart”, etc.). In the second step 201, the AI system generates an initial differential diagnosis. For example, the AI system may generate differential diagnosis of “#1 lung cancer, #2 pneumonia, #3 pulmonary infarct, #4 brain tumor”. The #4 would clearly be an erroneous differential diagnosis because the examination was a chest CT and the information field and patient specific information were both pertaining to the lung. The AI system could get tricked by phrases such as “central necrosis”, which can be used to describe both lung cancers and brain tumors. In the third step 202, exclusion criteria is applied based on certain fields to eliminate irrelevant or unlikely diagnoses. For example, if the study is a CT scan of the chest, if the AI lists differential diagnoses related to the brain when the field of interest is the lungs, then the differential diagnosis would be irrelevant and would be eliminated from the differential diagnosis list in the differential diagnosis icon box. Alternatively, if the study is a CT scan of the abdomen on a elderly man and if the field is kidney and the key term is renal mass and if the AI lists a diagnosis of Wilm's tumor, that differential diagnosis would be unlikely due to the fact that Wilm's tumors typically only occur in children and therefore would be eliminated from the differential diagnosis icon box. Therefore, the application of exclusion criteria would have value. In the fourth step 203, a differential diagnosis icon is displayed showing a more appropriate list of differential diagnoses.

FIG. 3 illustrates sample artificial intelligence algorithms including deep artificial neural networks and other machine learning algorithms. Inputs 300, hidden layers 301 and an output 302 are shown.

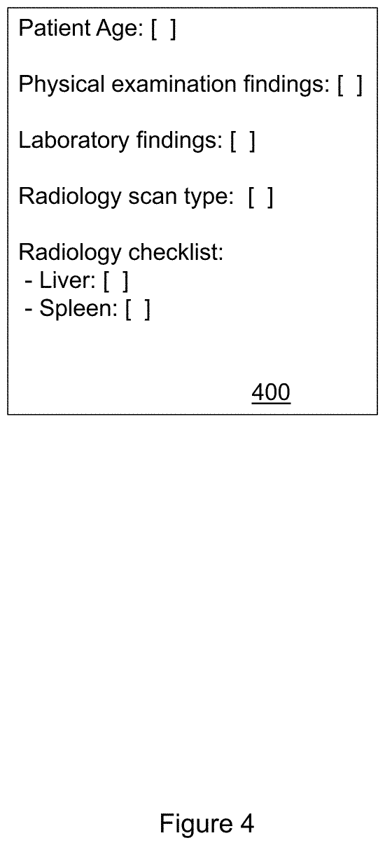

FIG. 4 illustrates generating information fields comprised of patient demographics, physical examination findings, laboratory findings, radiology scan type, radiology checklist item, radiology imaging findings, images or other patient specific features. The information fields 400 are shown. In this example, information fields are shown without being inside the bracket symbol “[ ]”. Example patient specific fields are designated by the bracket symbol “[ ]”. Inside the patient specific fields, findings terminology can be inputted.

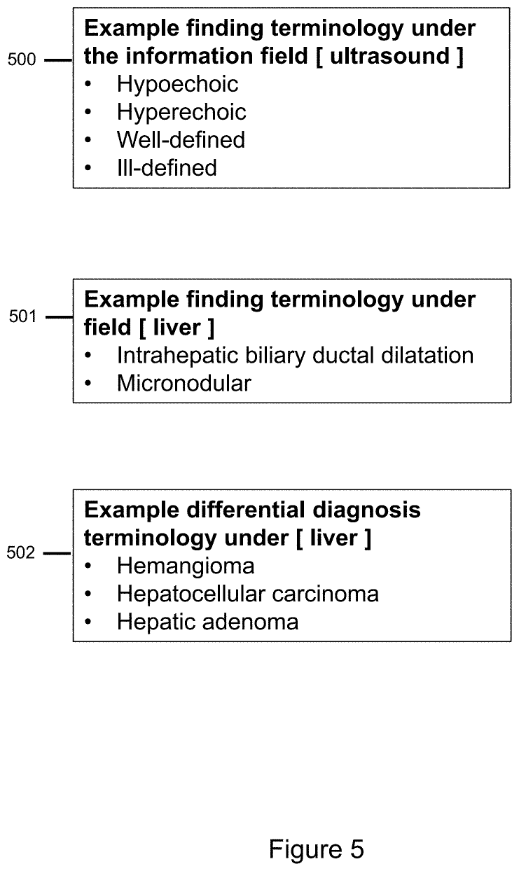

FIG. 5 illustrates an example list of terminology to be included in the information fields. Three examples are shown. In the first example 500, the finding terminology under the field ultrasound is shown and example terminology listed are as follows: hypoechoic; hyperechoic; well-defined; and, ill-defined. In the second example 501, the finding terminology under the field liver is shown and example terminology listed is as follows: intrahepatic biliary ductal dilitation; and, micronodular. In the third example 502, the differential diagnosis terminology under the field liver is as follows: hemangioma; hepatocellular carcinoma; and, hepatic adenoma.

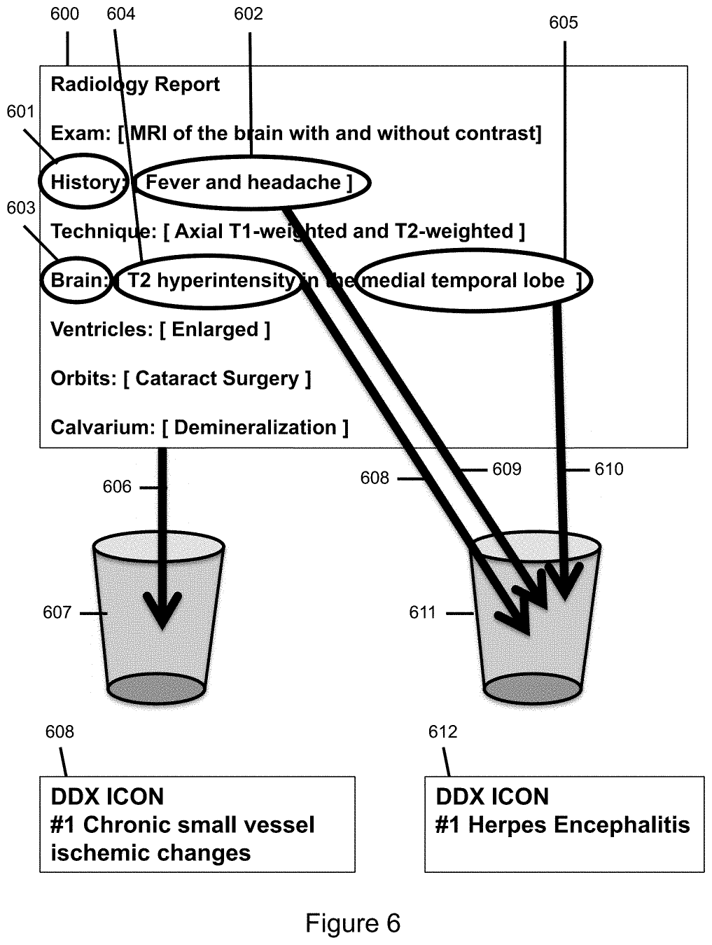

FIG. 6 illustrates computer inputted information and human inputted information for the AI algorithm. In this illustration, two scenarios are presented. In the first scenario, all information from all data fields is inputted into the AI algorithm to assist with the differential diagnosis. In the second scenario, the human selects only some data elements to be utilized to assist with the differential diagnosis. Patient specific information is selected from three information fields as follows: “fever and headache” patient specific information from the “history” information field; “T2 hyperintensity” patient specific information from the “brain” information field; and, “medial temporal lobe” patient specific information from the “brain” information field. Based on differences in the inputted data elements, the differential diagnosis determined by the AI algorithm may also be different. Controlling the inputs into an artificial intelligence algorithm may yield a differential diagnosis that is more useful to the clinician's thought process. It should also be noted that each individual sentence or phrase can be considered an information field inside of an information field. Highlighting or colored font may help the physician easily understand what went into the AI process. The sample radiology report 600 is shown. The information field “history” 601 contains the patient specific information “fever and headache” 601. The information field “brain” 603 contains the patient specific information “T2 hyperintensity” 604 and “medial temporal lobe” 605. In one scenario, the entire radiology report 600 is inputted 606 into a database 607 for AI analysis. The AI determines one or more differential diagnoses and rank orders these and presents this in a differential diagnosis icon 608, which states “DDX ICON #1 Chronic small vessel ischemic changes”. In another scenario, the human inputted the information field “history” 601 with the patient specific information “fever and headache” 601 is placed 609 into a database 611 for AI analysis. Also, the human inputted the information field “brain” 603 with the patient specific information “T2 hyperintensity” 604 is placed 608 into a database 611 for AI analysis. Also, the human inputted the information field “brain” 603 with the patient specific information “medial temporal lobe” 605 is placed 610 into a database 611 for AI analysis. The AI determines one or more differential diagnoses and rank orders these and presents this in a differential diagnosis icon 612, which states “DDX icon #1 Herpes Encephalitis”.

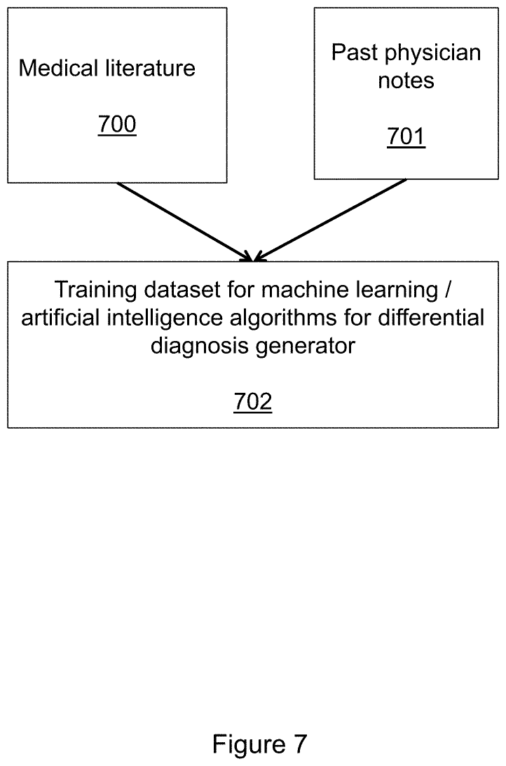

FIG. 7 illustrates utilization of materials for artificial intelligence algorithms. In this illustration, two sources are utilized as training datasets 702 for machine learning. The first source 700 is the example of the medical literature. For example, one of the open access sources that radiologists use is called radiopaedia.com. This source has a webpage for each diagnosis. Key data elements from this source can be used as a differential diagnosis generator. Alternatively, past physician notes 701 (e.g., past radiology reports) can also be used as the training dataset for machine learning/artificial intelligence.

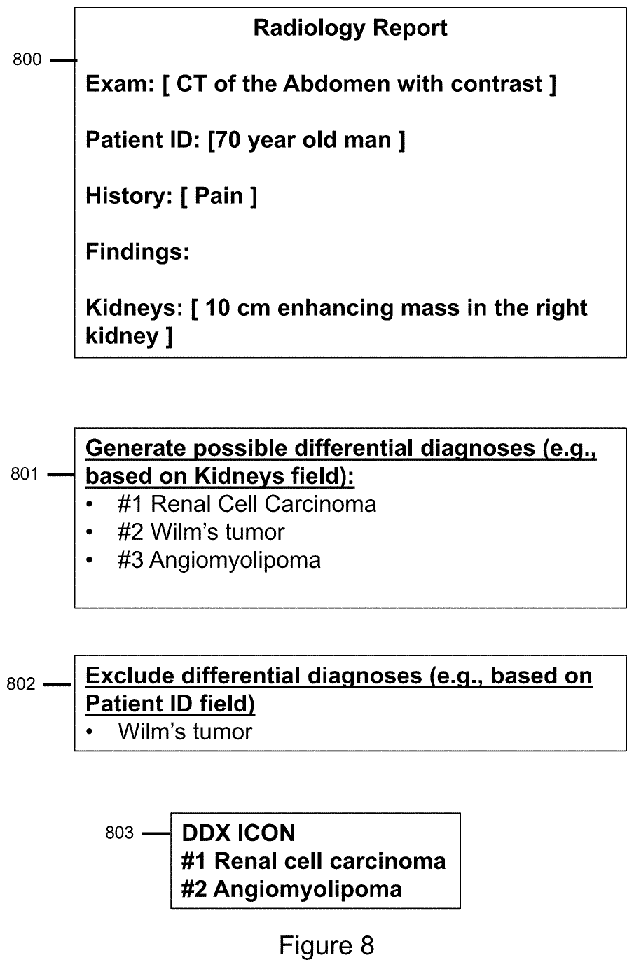

FIG. 8 illustrates a sample scenario wherein inclusion criteria and exclusion criteria are applied. A sample radiology report 800 is shown. In this scenario, the AI first generates a list of possible differential diagnoses based on the Kidneys field 801. As illustrated, this list includes renal cell carcinoma, Wilm's tumor, and angiomyolipoma. Next, the AI excludes differential diagnoses that are irrelevant or unlikely 802. As illustrated, this exclusion list includes Wilm's tumor, which typically only occurs in children and this patient is a 70-year-old man. Finally, the Differential diagnosis icon 803 is presented and shows only two of the three diagnoses.

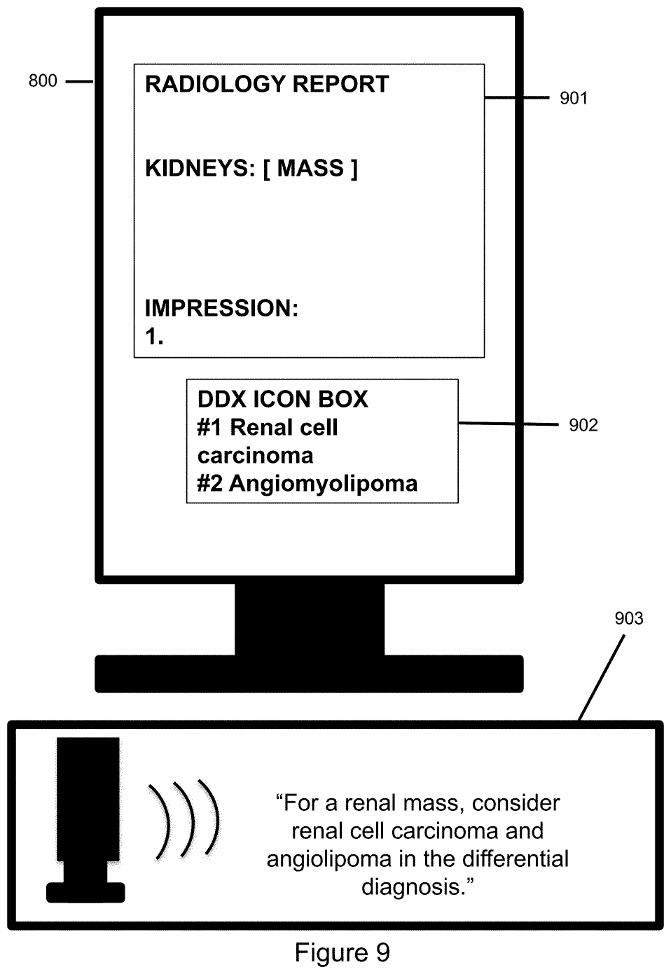

FIG. 9 illustrates visual representation to the user via visual representation or auditory representation. The computer monitor 900 is shown. The sample radiology report 901 is shown. The example visual representation illustrated is a pop-up box on the computer monitor 902 is shown. The example auditory representation 903 is a speaker projecting a sentence as follows: “for a renal mass, consider renal cell carcinoma and angiolipoma in the differential diagnosis.” is shown.

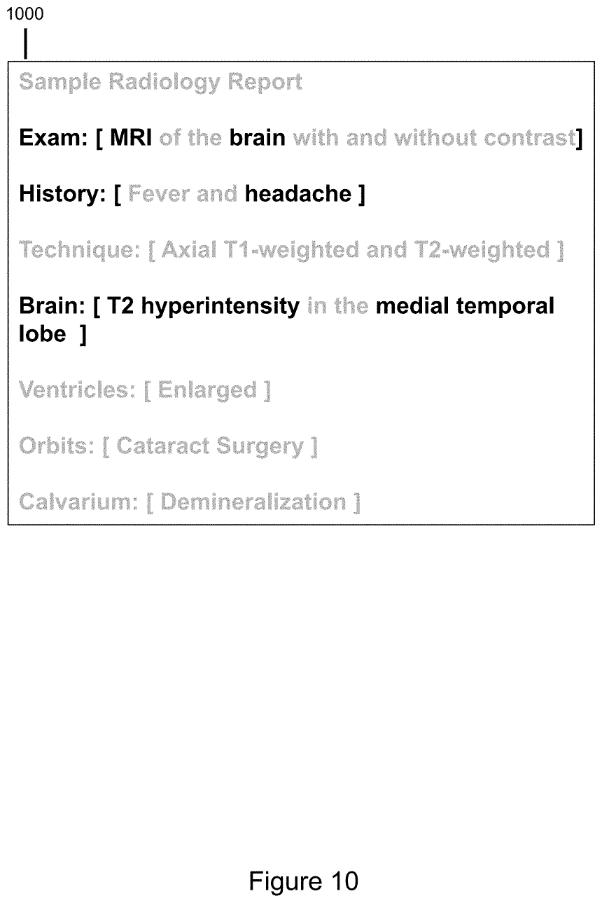

FIG. 10 illustrates human review of the pertinent item(s) considered by the AI algorithm. In this illustration 1000, the key fields and key words within each field that are used by the AI algorithm in generating the differential diagnosis are in black font and include the following: field “exam” and key words “MRI” and “brain”; field “history” and key word “headache”; and, field “brain” and key words/phrases “T2 hyperintensity” and “medial temporal lobe”. The key fields and key words within each field that are not used by the AI algorithm in generating the differential diagnosis are in gray font.

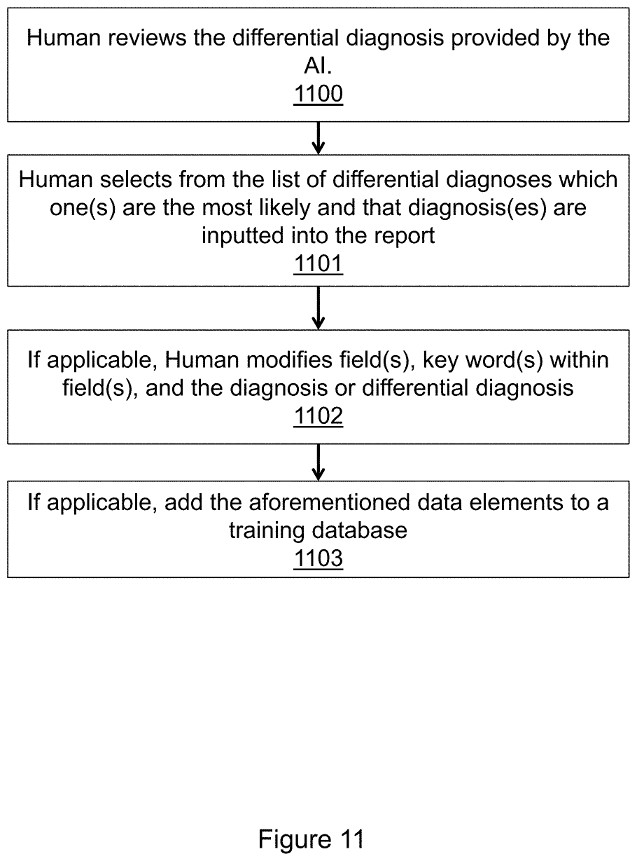

FIG. 11 illustrates human reviewing the differential diagnosis by the machine learning algorithm, selecting the diagnosis or differential diagnosis to be sent to the conclusion (aka, impression, assessment) section of the report. The flow diagrams do not depict the syntax of any particular programming language. Rather, the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables, are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order. The first step 1100 is for the human to review the differential diagnosis provided by the AI. The second step 1101 is for the human to select from the list of differential diagnoses which one(s) are the most likely and that diagnosis(es) are inputted into the report. The third step 1102, if applicable, is for the human to modify the field(s), key word(s) within field(s) and the diagnosis or differential diagnosis or order of the differential diagnosis. The fourth step 1103, if applicable, is to add the aforementioned data elements to a training database.

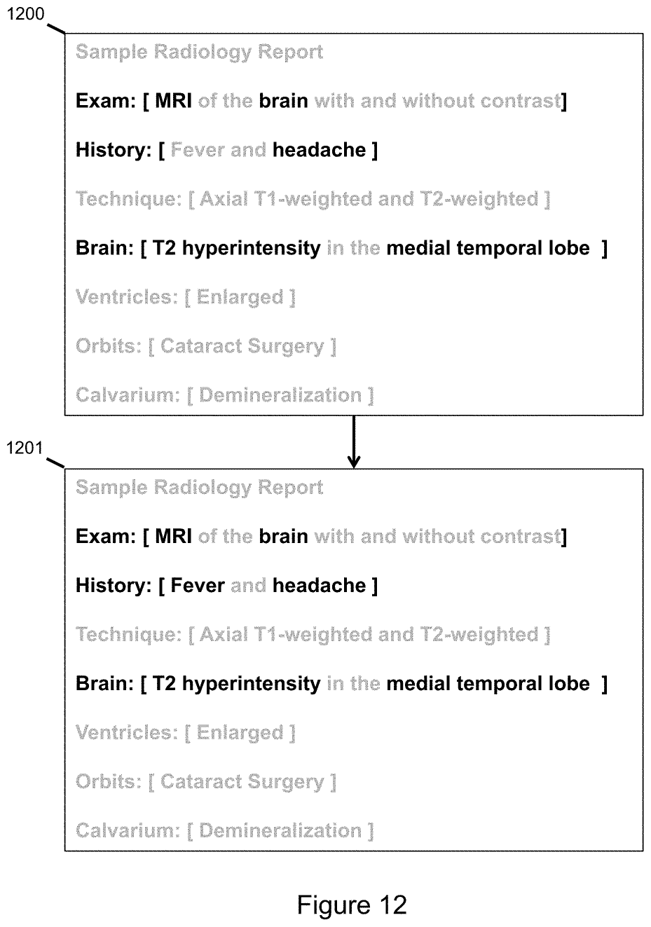

FIG. 12 illustrates modifying the patient specific information field(s) and/or the differential diagnosis(es). At the top of this illustration 1200 is a sample radiology report designating the key fields and key words used by the AI in determining the differential diagnosis. The key fields and key words within each field that are used by the AI algorithm in generating the differential diagnosis are in black font and include the following: field “exam” and key words “MM” and “brain”; field “history” and key word “headache”; and, field “brain” and key words/phrases “T2 hyperintensity” and “medial temporal lobe”. The key fields and key words within each field that are not used by the AI algorithm in generating the differential diagnosis are in gray font. At the bottom of this illustration 1201 is a sample radiology report designating human modified key fields and key words used by the AI in determining the differential diagnosis. One of the key words from one key field has been modified such that it should be included in the differential diagnosis. As illustrated, the word “fever” in the field “history” has been changed from gray font to black font. This denotes that it is important in determining the differential diagnosis. This is therefore a form of radiologist assisted machine learning. The radiologist can modify the importance of certain terms in generating a differential diagnosis. Weighting factors can be allocated. Other means of radiologist assisted machine learning can also be implemented.

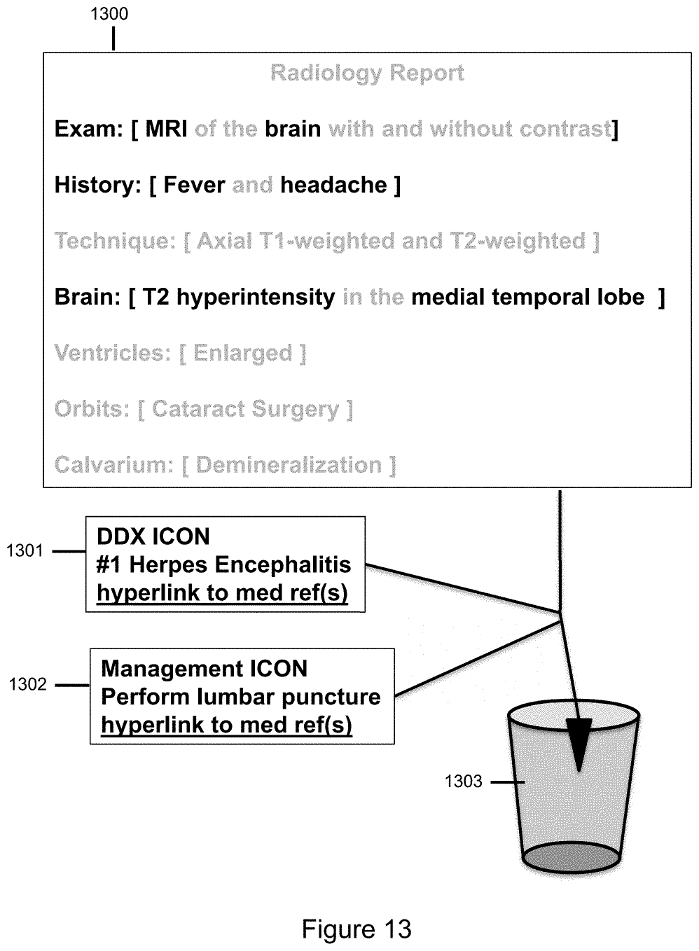

FIG. 13 illustrates updating the training dataset. As illustrated, the key fields with associated key words are modified by the human as shown in the top box sample radiology report 1300. The differential diagnosis is shown in the middle box DDX ICON 1301. The management recommendations are shown in the bottom box 1302 Management ICON. After any human modifications are performed, these elements are added to the training dataset 1303.

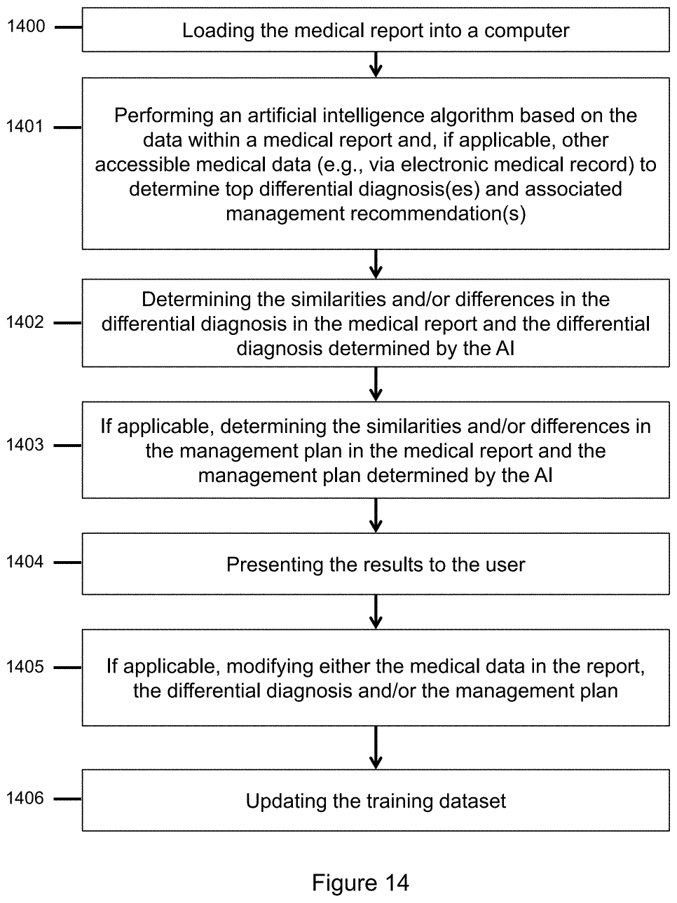

FIG. 14 illustrates a method of assessing the congruency between data within a medical report and the conclusion of a medical report. The flow diagrams do not depict the syntax of any particular programming language. Rather, the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables, are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order. Multiple key steps are shown. The first step 1400 is to load the medical report into a computer. The second step 1401 is to perform an artificial intelligence algorithm based on the data within a medical report and, if applicable, other accessible medical data (e.g., via electronic medical record) to determine top differential diagnosis(es) and associated management recommendation(s). The third step 1402 is to determine the similarities and/or differences in the differential diagnosis in the medical report and the differential diagnosis determined by the AI. The fourth step 1403, if applicable, is to determine the similarities and/or differences in the management plan in the medical report and the management plan determined by the AI. The fifth step 1404 is to present the results to the user. The sixth step 1405, if applicable, is to modify either the medical data in the report, the differential diagnosis and/or the management plan. The seventh step 1406 is to update the training dataset.

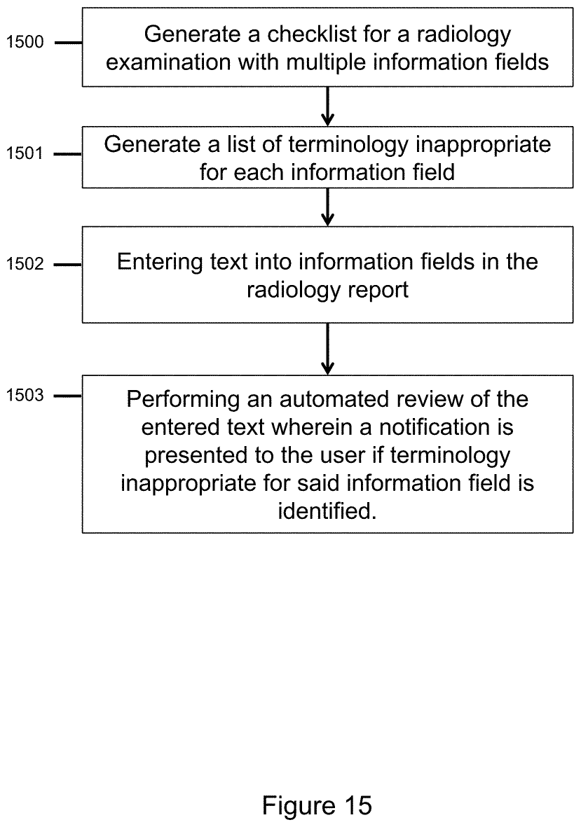

FIG. 15 illustrates a method to detect inappropriate terminology in a report. The flow diagrams do not depict the syntax of any particular programming language. Rather, the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables, are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order. The first step 1500 is to generate a checklist for a radiology examination with multiple information fields. The second step 1501 is to generate a list of terminology inappropriate for each information field. The third step 1502 is to enter text into information fields in the radiology report. The fourth step 1503 is to perform an automated review of the entered text wherein a notification is presented to the user if terminology inappropriate for said information field is identified.

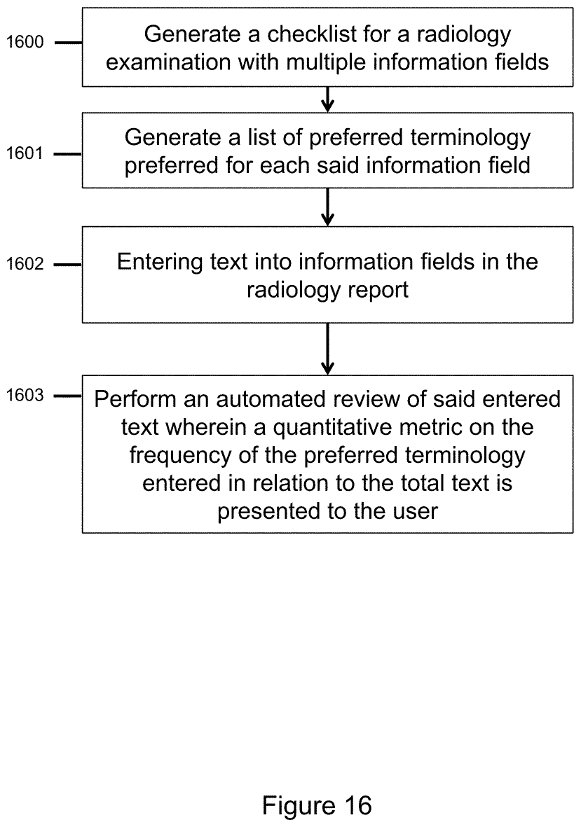

FIG. 16 illustrates a method to quantitatively assess report performance. The flow diagrams do not depict the syntax of any particular programming language. Rather, the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables, are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order. The first step 1600 is to generate a checklist for a radiology examination with multiple information fields. The second step 1601 is to generate a list of preferred terminology preferred for each said information field. For example, for the kidney field, terminology could include “parenchyma”, “stone”, “hydronephrosis”, “delayed nephrogram”. The third step 1602 is to enter text into information fields in the radiology report. The fourth step 1603 is to perform an automated review of said entered text wherein a quantitative metric on the frequency of the preferred terminology entered in relation to the total text is presented to the user. A radiologist needs to be efficient to improve medical care and prevent information overload. This metric will help maintain efficiency in reporting.

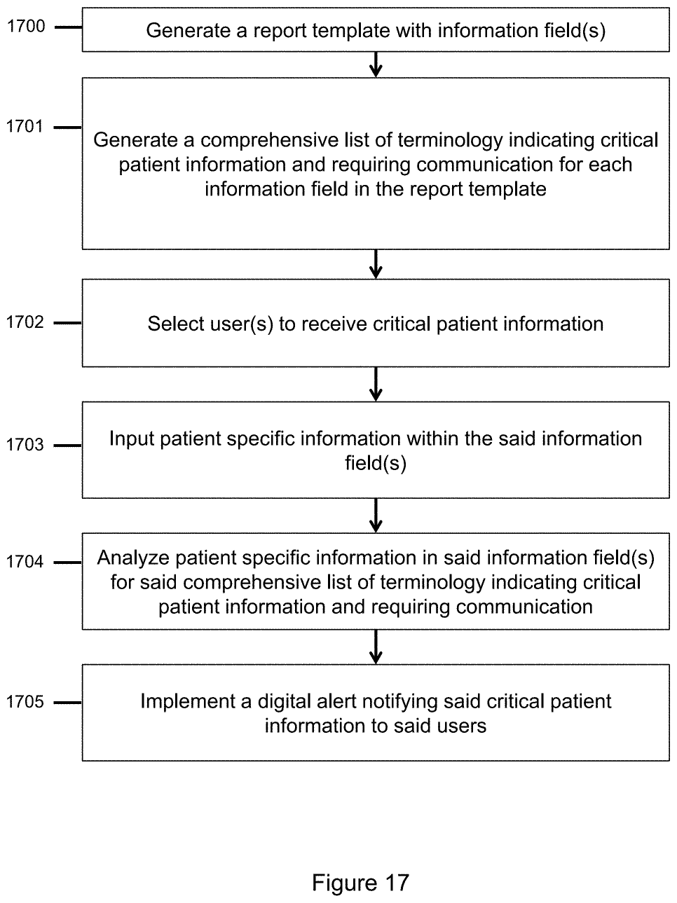

FIG. 17 illustrates a method of assured communication of critical patient information during medical reporting. The flow diagrams do not depict the syntax of any particular programming language. Rather, the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables, are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order. The first step 1700 is to generate a report template with information field(s). The second step 1701 is to generate a comprehensive list of terminology indicating critical patient information and requiring communication for each information field in the report template. The third step 1702 is to select user(s) to receive critical patient information. The fourth step 1703 is to input patient specific information within the said information field(s). The fifth step 1704 is to analyze patient specific information in said information field(s) for said comprehensive list of terminology indicating critical patient information and requiring communication. The sixth step 1705 is to implement a digital alert notifying said critical patient information to said users. An option would be to alert the radiologist first and then allow the radiologist to pass the critical information to the ordering physician.

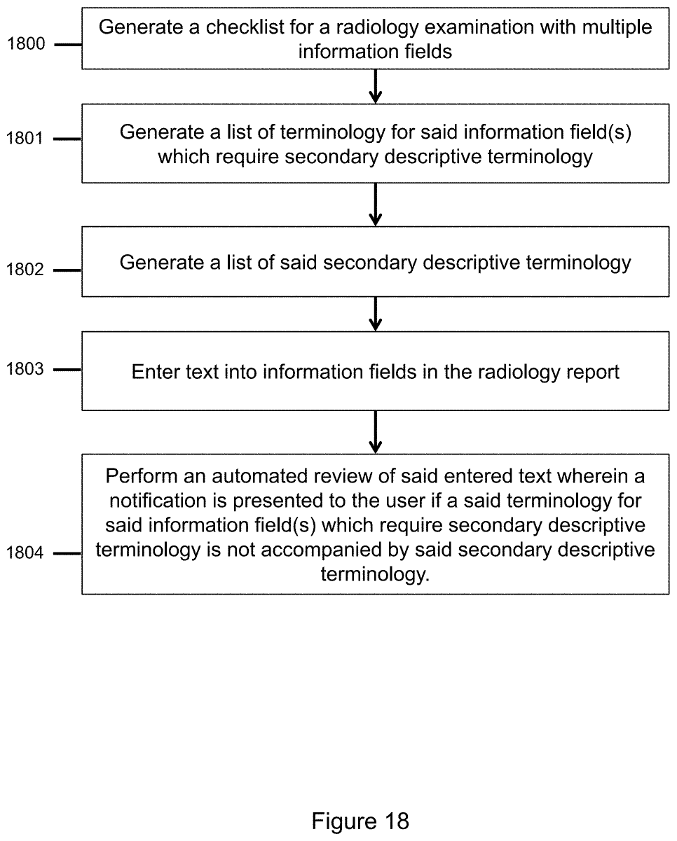

FIG. 18 illustrates a method to improve medical reporting by alerting a user when secondary descriptive terminology should be inputted. The flow diagrams do not depict the syntax of any particular programming language. Rather, the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables, are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order. The first step 1800 is to generate a checklist for a radiology examination with multiple information fields. The second step 1801 is to generate a list of terminology for said information field(s) which require secondary descriptive terminology. The third step 1802 is to generate a list of said secondary descriptive terminology. The fourth step 1803 is to enter text into information fields in the radiology report. The fifth step 1804 is to perform an automated review of said entered text wherein a notification is presented to the user if a said terminology for said information field(s) which require secondary descriptive terminology is not accompanied by said secondary descriptive terminology.

Several features, aspects, embodiments, and implementations have been described. Nevertheless, it will be understood that a wide variety of modifications and combinations may be made without departing from the scope of the inventive concepts described herein. Accordingly, those modifications and combinations are within the scope of the following claims.

What is claimed is:

1. A method of assisting a medical professional develop a differential diagnosis comprising:
generating a report template with at least one information field;
generating a comprehensive list of possible differential diagnoses for each information field in the report template;
inputting patient specific information within the at least one information field; and
performing an artificial intelligence program to yield a list of top output differential diagnoses wherein the list of top output differential diagnoses is selected from the comprehensive list of possible differential diagnoses.

2. The method of claim 1 comprising running artificial intelligence algorithms including deep artificial neural networks and machine learning algorithms.

3. The method of claim 1 comprising medical personnel reporting including diagnostic radiologists, surgeons, primary care physicians and specialists.

4. The method of claim 1 wherein generating the report template with information fields comprises including patient demographics, physical examination findings, laboratory findings, radiology scan type, radiology checklist item, radiology imaging findings, images and other patient specific features.

5. The method of claim 1 wherein generating the comprehensive list of possible differential diagnoses for each information field in the report template comprises including medical textbooks, ICD codes and expert consensus.

6. The method of claim 1 wherein inputting patient specific information comprises including computer inputted information and human inputted information.

7. The method of claim 1 wherein performing the artificial intelligence program to yield the list of top output differential diagnoses comprises utilizing single fields or combining multiple fields.

8. The method of claim 1 wherein performing the artificial intelligence program to yield a list of top output differential diagnoses comprises selecting key items within a field such as numbers, single words or combinations of multiple words.

9. The method of claim 1 wherein performing the artificial intelligence program comprises running deep artificial neural networks and other machine learning algorithms.

10. The method of claim 1 wherein performing the artificial intelligence program comprises utilizing materials including training datasets and medical references.

11. The method of claim 10 wherein machine learning includes generating and applying inclusion criteria resulting in a list of all possible differential diagnoses based one or more fields.

12. The method of claim 10 wherein machine learning includes generating and applying exclusion criteria where non-relevant differential diagnoses and unlikely differential diagnoses are eliminated from display.

13. The method of claim 10 wherein machine learning includes generating the differential diagnoses based on patient specific information from at least two information fields.

14. The method of claim 1 wherein outputting a differential diagnosis by the artificial intelligence algorithm comprises one of the group of: visually representing to the user via a pop-up box icon on the computer; and, auditorily representing via an audible voice recording.

15. The method of claim 1 wherein human review comprises one of the group of: the pertinent item(s) being considered by the artificial intelligence algorithm; weighting factors; past medical reports; and, other medical references.

16. The method of claim 1 wherein reviewing the differential diagnosis by the artificial intelligence algorithm comprises selecting the diagnosis or differential diagnosis to be sent to the conclusion (aka, impression, assessment) section of the report.

17. The method of claim 1 comprising performing a second review of the medical examination, revising the patient specific information fields or the impression section of the report the differential diagnosis and changing at least one item of one field.

18. The method of claim 1 comprising providing a hyperlink to medical reference materials supporting the differential diagnosis provided.

19. The method of claim 1 comprising updating the training dataset by adding new information including the patient specific information fields with an associated differential diagnosis and management recommendations.

20. A method of assessing the congruency between data within a medical report and the conclusion of a medical report comprising:
loading the medical report into a computer; and
performing an artificial intelligence program to determine the congruency between the patient specific information within at least one information field and an impression section of the report.

21. The method of claim 20 comprising alerting a medical professional of any discordance between the patient specific information within the at least one information field and the impression section of the report.

22. A method of precision radiology reporting comprising:
generating a checklist for a radiology examination with multiple information fields;
generating a list of terminology inappropriate for said information fields;
entering text into information fields in the radiology report; and
performing an automated review of said entered text wherein a notification is presented to the user if terminology inappropriate for said information field is identified.

23. A method of precision radiology reporting comprising:
generating a checklist for a radiology examination with multiple information fields;
generating a list of terminology for ones of the information fields that require secondary descriptive terminology;
generating a list of said secondary descriptive terminology;
entering text into the information fields in the radiology report; and
performing an automated review of said entered text wherein a notification is presented to the user if a said terminology for said information fields which require secondary descriptive terminology is not accompanied by said secondary descriptive terminology.

24. A method of characterizing a radiology report comprising:
generating a checklist for a radiology examination with multiple information fields;
generating a list of preferred terminology preferred for said information fields;
entering text into information fields in the radiology report; and
performing an automated review of said entered text wherein a quantitative metric on the frequency of the preferred terminology entered in relation to the total text is presented to the user.

25. A method of assured communication of critical patient information during medical reporting comprising:
generating a report template with at least one information field;
generating a comprehensive list of terminology indicating critical patient information and requiring communication for each information field in the report template;
selecting at least one user to receive critical patient information;
inputting patient specific information within the said at least one information field;
analyzing said patient specific information in said at least one information field for said comprehensive list of terminology indicating critical patient information and requiring communication; and
implementing a digital alert notifying said critical patient information to said users.

26. The method of claim 25 comprising electronically providing notification to a first user when a second user receives said digital alert.
